(19) United States
(12) Patent Application Publication
(10) Pub. No.: US 2022/0226383 A1
(43) Pub. Date: Jul. 21, 2022

(54) METHOD OF PRESERVING CELLS FOR THERAPEUTIC USE

(71) Applicant: LABORATOIRE FRANÇAIS DU FRACTIONNEMENT ET DES BIOTECHNOLOGIES, LES ULIS (FR)

(72) Inventors: Joffrey DE LARICHAUDY, MALAKOFF (FR); Sandy CAZALON NEMORIN, LONGJUMEAU (FR)

(21) Appl. No.: 17/293,867

(22) PCT Filed: Nov. 14, 2019
(86) PCT No.: PCT/EP2019/081339

§ 371 Date: May 13, 2021

(30) Foreign Application Priority Data
Nov. 15, 2018 (FR) 1860544

(51) Int. Cl. A61K 35/28; A61K 47/26; A61K 47/24; A61K 47/18; A61K 47/22; A61K 47/46; A61K 35/34; A61K 47/12; A61K 9/00

(52) U.S. Cl. CPC A61K 35/28; A61K 47/26; A61K 47/24; A61K 47/183; A61K 9/0019; A61K 47/18; A61K 47/46; A61K 35/34; A61K 47/12; A61K 47/22

(57) ABSTRACT

Disclosed is a composition including, in a physiologically acceptable medium: a) at least one saccharide; b) at least one vitamin; c) at least one amino acid; d) at least one antioxidant; and e) cells for therapeutic use. The composition has a pH between 7.0 and 8.5, inclusive, and preferably between 7.0 and 8.3. Also disclosed is a method of preserving a sample of cells for therapeutic use, including at least one step of mixing the sample of cells for therapeutic use with the ingredients a) to d) above and a physiologically acceptable medium, the composition that is obtained having a pH between 7.0 and 8.5, inclusive, and preferably between 7.0 and 8.3.

### CROSS-REFERENCE TO RELATED APPLICATIONS

This application is the U.S. national phase of International Application No. PCT/EP2019/081339 filed Nov. 14, 2019 which designated the U.S. and claims priority to FR 1860544 filed Nov. 15, 2018, the entire contents of each of which are hereby incorporated by reference.

### BACKGROUND OF THE INVENTION

### Field of the Invention

The present invention relates to a composition comprising, in a physiologically acceptable medium:
a) at least one saccharide;
b) at least one vitamin;
c) at least one amino acid;
d) at least one antioxidant; and
e) cells for therapeutic use,

the said composition having a pH between 7.0 and 8.5, inclusive, and preferably between 7.0 and 8.3.

The present invention also relates to a cell preservation method for preserving a sample of cells for therapeutic use, comprising at least one mixing step of mixing the sample of cells for therapeutic use with:
a) at least one saccharide;
b) at least one vitamin;
c) at least one amino acid;
d) at least one antioxidant; and
a physiologically acceptable medium;
the composition thus obtained having a pH between 7.0 and 8.5, preferably between 7.0 and 8.3.

The present invention also relates to the use of a composition comprising, in a physiologically acceptable medium:
a) at least one saccharide;
b) at least one vitamin;
c) at least one amino acid;
d) at least one antioxidant;

and having a pH between 7.0 and 8.5, preferably between 7.0 and 8.3, in order to ensure preservation of at least one sample of cells for therapeutic use.

### Description of the Related Art

The preservation of biological samples, that are in particular intended for therapeutic use, is a crucial issue for the pharmaceutical industry.

In general, the biological material is preserved by means of freezing of the said material, in an appropriate support carrier, such as a tube or an ampoule made of glass or plastic material (generally referred to as “straw” or freezing tube in the field of cryopreservation), the said support carrier being suitable for long-term storage and at low temperatures; this is cryopreservation.

However, cryopreservation presents a certain number of problems and technical constraints. In particular, cell damage can occur during thawing, resulting in apoptosis or bursting of the cells. In addition, the survival of cryopreserved cells may depend on the conditions and techniques used at the time of freezing. For the majority of mammalian cells, as is the case with cell therapy products and medicaments, it is additionally also essential to use cryoprotectants in order to preserve cell integrity and functionality.

Currently, the production on an industrial scale (European or worldwide in particular) of cell therapy products poses new problems, such as the stability of the finished product administered and the variability linked to the starting biological material.

In most cases, the finished product is packaged:
fresh, in a liquid medium (such as albumin or saline solution) with a preservation period limited to a few hours; or
frozen, in a simple formulation based on dimethyl sulfoxide (DMSO), which is not very stable and not very efficacious in the long term. The not insignificant quantity of dead cells, and debris with potentially immunogenic effects, as also the toxicity to the patient of the commonly used excipients all represent limiting factors. Most of the time, washing of the cells prior to administration in order to remove these toxic elements (whether or not biological) is indicated. These solutions are therefore not compatible with industrial distribution, and offer very little flexibility in terms of administration and storage.

There is therefore a need for the development of a cell therapy product and/or medicament that is stable over the long term (i.e. for periods of one, two, three days, up to a week), and which is easy to use, directly injectable and nontoxic.

In addition, such a cell therapy product and/or medicament should exhibit well preserved cell viability and functionality.

### SUMMARY OF THE INVENTION

The present invention makes it possible to address this need. In fact, the composition according to the invention makes it possible to obtain cell therapy products comprising cells intended for therapeutic applications, that are ready for use/administration (i.e. ready to be injected without washing, which serves to avoid all additional handling or manipulations resulting in a drop in viability and a loss of cells), easy to use and non-toxic. In addition, the composition according to the invention makes it possible to preserve the viability of cells intended for therapeutic use, with the functionality thereof being maintained (i.e. for periods of one, two, three days, up to a week).

The present invention relates to a composition comprising, in a physiologically acceptable medium:
a) at least one saccharide;
b) at least one vitamin;
c) at least one amino acid;
d) at least one antioxidant, and
e) cells for therapeutic use;

the said composition having a pH between 7.0 and 8.5, preferably between 7.0 and 8.3.

The present invention also relates to a cell preservation method for preserving a sample of cells for therapeutic use, comprising at least one mixing step of mixing the sample of cells for therapeutic use with:
a) at least one saccharide;
b) at least one vitamin;
c) at least one amino acid;
d) at least one antioxidant, and
a physiologically acceptable medium;
the composition thus obtained having a pH between 7.0 and 8.5, preferably between 7.0 and 8.3.

The present invention also relates to the use of a composition comprising, in a physiologically acceptable medium:
a) at least one saccharide;
b) at least one vitamin;
c) at least one amino acid;
d) at least one antioxidant;

and having a pH between 7.0 and 8.5, preferably between 7.0 and 8.3, for preserving at least one sample of cells for therapeutic use.

The composition according to the invention therefore comprises, in a physiologically acceptable medium:
a) at least one saccharide;
b) at least one vitamin;
c) at least one amino acid;
d) at least one antioxidant, and
e) cells for therapeutic use;

In addition, the composition has a pH of between 7.0 and 8.5, preferably between 7.0 and 8.3.

According to one embodiment, the composition according to the invention is directly injectable into the patient.

The term “physiologically acceptable medium” is understood to refer to an aqueous medium containing electrolytes. The electrolytes are for example, salts of sodium, potassium, magnesium and/or calcium, with anions of such types as chloride, acetate, carbonate, hydrogen carbonate, hydroxide or citrate. Preferably, the physiologically acceptable medium is an aqueous medium comprising at least sodium chloride, potassium chloride and calcium chloride. According to this preceding sentence, preferably, the physiologically acceptable medium comprises in addition sodium acetate and trisodium citrate.

The sodium is preferably present in the composition according to the invention in a concentration of between 130 and 200 mmol/L, preferably between 135 and 190 mmol/L, preferably between 138 and 188 mmol/L.

The potassium is preferably present in the composition according to the invention in a concentration of between 0.5 and 5.0 mmol/L, preferably between 1.0 and 4.5 mmol/L, preferably between 1.5 and 4.0 mmol/L.

The calcium is preferably present in the composition according to the invention in a concentration of between 0.01 and 10 mmol/L, preferably between 0.01 and 1 mmol/L, preferably between 0.01 and 0.05 mmol/L.

The chloride is preferably present in the composition according to the invention in a concentration of between 40 and 110 mmol/L, preferably between 70 and 105 mmol/L, preferably between 65 and 100 mmol/L.

The magnesium is preferably present in the composition according to the invention in a concentration of between 0 and 5 mmol/L, preferably between 0.5 and 4.5 mmol/L, preferably between 1 and 3.5 mmol/L.

The physiologically acceptable medium preferably comprises at least one hydrogencarbonate salt (HCO₃⁻) (also known as bicarbonate). Preferably the physiologically acceptable medium comprises sodium hydrogencarbonate.

Preferably, the physiologically acceptable medium is such that the composition that contains it has a pH of between 7.0 and 8.5, preferably between 7.0 and 8.3. In particular, the HCO₃⁻ ions are present therein in order to adjust the pH so as to be within this range of values.

Preferably, the bicarbonate is present in the composition according to the invention at a concentration of between 20 and 100 mmol/L, preferably between 20 and 80 mmol/L, preferably between 20 and 60 mmol/L, preferably between 20 and 55 mmol/L.

Preferably, the composition according to the invention has an osmolarity of between 250 and 400 mOsm/L, preferably between 260 and 390 mOsm/L, preferably between 280 and 320 mOsm/L.

The composition according to the invention comprises at least one saccharide (compound a)). The saccharide enhances the survival and function of cells by preserving the osmotic balance. A saccharide moiety penetrates the cells and serves the purpose of stabilising the membrane structures. The saccharide is preferably selected from among monosaccharides, disaccharides and trisaccharides.

The monosaccharides are preferably selected from glucose, galactose, fructose and mannose. Preferably, the saccharide is glucose.

The disaccharide preferably has the formula A-B, in which A and B are each independently selected from glucose, fructose and mannose. The saccharide is preferably a disaccharide. The disaccharide is preferably a glucose dimer. More preferentially, the disaccharide is selected from trehalose and sucrose.

The trisaccharides are preferably selected from raffinose (trimer of galactose, glucose and fructose), maltotriose and isomaltotriose (trimer of glucose).

The saccharide is preferably present in the composition according to the invention in a concentration of between 10 and 20 mmol/L, preferably between 10 and 15 mmol/L, preferably between 12.5 and 15 mmol/L.

The composition according to the invention comprises at least one vitamin (compound b)). The one or more vitamin(s) is(are) selected in particular from among the vitamins B1 (thiamine), B2 (riboflavin), B4 (choline), B5 (panthotenic acid), B6 (pyridoxal), B7 (inositol), B9 (folic acid), PP (nicotinamide) and the mixtures thereof.

Preferably, the composition according to the invention comprises a mixture of vitamins B1, B2, B4, B5, B6, B7, B9 and PP. Such a mixture of vitamins is in particular marketed by Thermo Fisher under the reference Gibco® MEM Vitamin Solution (100×).

The one or more vitamin(s) is(are) preferably present in the composition according to the invention in a concentration of between 0.1 and 100 mg/L, preferably between 0.5 and 90 mg/L, preferably between 1 and 80 mg/L, preferably between 1.5 and 70 mg/L, preferably between 2 and 60 mg/L, preferably between 2.5 and 50 mg/L, preferably between 3 and 40 mg/L, preferably between 3.5 and 30 mg/L, preferably between 4 and 20 mg/L, preferably between 4.5 and 20 mg/L, preferably between 5 and 10 mg/L.

The composition according to the invention comprises at least one amino acid (compound c)). Preferably, the one or more amino acid(s) is(are) selected from among glutamine, alanyl-glutamine, tryptophan, lysine, methionine, phenylalanine, threonine, valine, leucine and isoleucine, arginine, histidine, tyrosine, cysteine and the mixtures thereof.

Preferably, the composition according to the invention comprises at least one mixture of glutamine and alanyl-glutamine, in particular a mixture of L-glutamine and L-alanyl-L-glutamine.

Preferably, the composition according to the invention comprises essential amino acids. An essential amino acid is an amino acid which cannot be synthesised de novo by the organism or body (generally human) or which is synthesised at an insufficient rate, and must therefore be provided through nutrition intake, a necessary condition for the proper functioning of the organism or body.

In humans, there happens to be eight essential amino acids: tryptophan, lysine, methionine, phenylalanine, threonine, valine, leucine and isoleucine.

The composition according to the invention preferably comprises the eight essential amino acids mentioned above, as well as arginine, histidine, tyrosine and cysteine. Such a mixture of amino acids is notably marketed by Thermo Fisher under the reference Gibco® MEM Amino Acids 50×.

The amino acids are preferably present in the composition according to the invention in a concentration of between 10 and 700 mg/L, preferably between 50 and 700 mg/L, preferably between 100 and 700 mg/L, preferably between 150 and 700 mg/L, preferably between 200 and 700 mg/L, preferably between 250 and 700 mg/L, preferably between 300 and 700 mg/L, preferably between 300 and 600 mg/L.

The composition according to the invention preferably comprises a mixture of the eight essential amino acids mentioned above, as also arginine, histidine, tyrosine, cysteine, glutamine and alanyl-glutamine.

Preferably, the composition according to the invention comprises cysteine.

The composition according to the invention comprises at least one antioxidant (compound d)). The term “antioxidant” is understood to refer to any compound which serves the purpose of slowing down or preventing the oxidation caused by an oxidising agent which can lead to the production of free radicals. In the composition of the present invention, the antioxidant provides the means to protect the cells from oxidative stress and therefore to maintain or enhance their viability.

Preferably, the composition according to the invention comprises at least one antioxidant selected from among glutathione, vitamin C, vitamin E, vitamin A, L-cysteine, or the coenzyme Q10.

Preferably, the composition according to the invention comprises glutathione.

Preferably, the one or more antioxidant(s) is(are) present in the composition according to the invention in a concentration of between 0.5 and 2 g/L, preferably between 0.5 and 1.5 g/L, preferably between 0.8 and 1 g/L.

Preferably, the composition according to the invention further comprises a platelet lysate. The platelet lysate is preferably present in the composition according to the invention in a concentration of between 5% and 30% by volume, preferably between 15% and 25% by volume relative to the total volume of composition.

Preferably, the platelet lysate comprises at least one growth factor selected from among transforming growth factor beta-1 (TGF-beta1), endothelial growth factor (EGF), platelet-derived growth factor-AB (PDGF-AB), insulin growth factor-1 (IGF-1), vascular endothelial growth factor (VEGF), basic fibroblast growth factor (bFGF), and the mixtures thereof.

Preferably, the composition according to the invention is substantially free of dextran. In respect of the composition, the term “substantially free” is understood to indicate that this composition contains less than 10% by weight, preferably less than 5% by weight, preferably less than 3% by weight, preferably less than 1% by weight of dextran. Preferably, the composition according to the invention does not contain dextran.

Preferably, the composition according to the invention comprises, in an aqueous medium containing electrolytes:
a) glucose;
b) a mixture of vitamins B1, B2, B4, B5, B6, B7, B9 and PP;
c) a mixture of glutamine, alanyl-glutamine, tryptophan, lysine, methionine, phenylalanine, threonine, valine, leucine, isoleucine, arginine, histidine, tyrosine and cysteine; and
d) glutathione.

The composition according to the invention is of particularly beneficial interest, and is designed to preserve at least one sample of cells intended for therapeutic use. Indeed, the compounds a) to d) used in the composition provide the ability to preserve the cells intended for therapeutic use, in a durable and effective manner.

The term “preservation” or “conservation”, is understood to indicate that the cell viability after 24 hours of incubation in the composition of the invention at a temperature of between +1° C. and +5° C. inclusive, preferably at a temperature of +4° C., is between 85% and 100%; the cell viability after 48 hours of incubation in the composition of the invention at a temperature of between +1° C. and +5° C. inclusive, preferably at a temperature of +4° C., is between 80% and 100%; the cell viability after 72 hours of incubation in the composition of the invention at a temperature of between +1° C. and +5° C. inclusive, preferably at a temperature of +4° C., is between 70% and 100%.

In one embodiment of the invention, the cell viability measurement is performed directly on the cells in the composition of the invention by flow cytometry after labelling of the cells with propidium Iodide (PI) which is a marker for cell viability. In a second embodiment, the cell viability measurement is performed directly on the cells in the composition of the invention by performing a count on Malassez counting chambers after labelling of the cells with trypan blue. In a third embodiment, the cell viability measurement is performed directly on the cells in the composition of the invention by counting with an automated cell counter such as Nucleocounter after labelling with Acridine Orange (cell marker) and DAPI (4′,6-diamidino-2-phenylindole—cell mortality marker).

The term “cells (intended) for therapeutic use” is understood to refer to cells that in and of themselves constitute the therapeutic product, and which are administered to the patient.

These cells are distinct from cells which are cultured for the production of biological medicinal products, such as for example Chinese hamster ovary (CHO), human embryonic kidney (HEK) cell lines, or rat myeloma cell line YB2/0, and are not intended to be administered to the patient.

Among the cells for therapeutic use, in particular mention may be made of innovative therapy medicinal products or cell therapy products.

The cells for therapeutic use (compounds d)) are preferably selected from among:
immune cells, such as natural killer (NK) cells; monocytes; B lymphocytes; T lymphocytes, whether natural or genetically modified, such as regulatory T lymphocyte cells (Tregs), tumour infiltrating T lymphocytes, cytotoxic T lymphocytes, helper T lymphocytes (or helpers) and T lymphocytes having a chimeric antigen receptor (CAR);
myoblasts in particular human myoblasts;
hematopoietic stem cells;
mesenchymal stem cells;
cardiac cells;
fibroblasts; and
all other natural or genetically modified cells.

The NK cells (or NK lymphocytes) are cells of the innate immune system. These are non-T lymphocytes (CD3−), non-B lymphocytes (CD19−), characterised in humans by the markers CD56, CD16 and NK.

The monocytes are leukocytes which evolve into macrophages, dendritic cells or osteoclasts.

The B lymphocytes are the immune cells responsible for the production of antibodies.

The regulatory T cells are a subpopulation of CD4+ T cells, which inhibit the proliferation of other effector T cells.

Cytotoxic T cells are a subpopulation of CD8+ T cells, which destroy infected cells.

Helper T cells are a subpopulation of CD4+ T cells, which are intermediaries of the immune response.

Finally, T lymphocytes that have a chimeric antigen receptor (CAR), also known as chimeric antigen receptor T cells (CAR-T), correspond to a particular cellular engineering technology. These are T lymphocytes that express a chimeric antigen receptor. CAR-T cells are capable of killing cancer cells, by recognising and binding to the tumour antigen present on the said cancer cells.

The sample of cells for therapeutic use may be derived from the patient to be treated (in this case the patient and the donor are the same person), by biopsy or blood sample collection. In this case, the composition that is obtained and conserved will be administered to this very same patient: this is an autologous product.

Alternatively, the sample of cells for therapeutic use may be derived from another source (i.e. another individual, cell engineering), in particular by biopsy or blood sample collection. In this case, the composition that is obtained and conserved will be administered to a patient to be treated who is other than the donor: this is an allogenic product.

Preferably, the composition according to the invention comprises a concentration of cells of between 2 and 300 M cells/mL, preferably between 10 and 200 M cells/mL, preferably between 20 and 100 M cells/mL.

The present invention also relates to a cell preservation method for preserving a sample of cells for therapeutic use, comprising at least one mixing step of mixing the sample of cells for therapeutic use with:
a) at least one saccharide;
b) at least one vitamin;
c) at least one amino acid;
d) at least one antioxidant;
a physiologically acceptable medium;
the composition thus obtained having a pH between 7.0 and 8.5, preferably between 7.0 and 8.3.

In this method, the mixing step of mixing the sample of cells for therapeutic use with the various compounds described above is typically carried out by dilution. The mixing can be done at a temperature of between +1° C. and +20° C. inclusive, preferably between +2° C. and +20° C. inclusive, preferably between +2° C. and +15° C. inclusive, preferably between +2° C. and +10° C. inclusive, preferably between +2° C. and +6° C. inclusive, preferably at 4° C.

Preferably, the sample of cells for therapeutic use is, for its part, previously cultured in vitro, in an appropriate culture medium. Thereafter it undergoes centrifugation, then the supernatant is removed and the pellet is suspended in a mixture of physiologically acceptable medium and the compounds a) to d) described above, in order to obtain a composition having a pH of between 7.0 and 8.5, preferably between 7.0 and 8.3.

The sample can then be conserved, being kept at a temperature of between +1° C. and +20° C. inclusive, preferably between +2° C. and +20° C. inclusive, preferably between +2° C. and +15° C. inclusive, preferably between +2° C. and +10° C. inclusive, preferably between +2 and +6° C. inclusive, preferably at +4° C.

The invention is illustrated by means of the following examples, which are not in any way intended to be limiting.

### DESCRIPTION OF THE PREFERRED EMBODIMENTS

### Examples

### Example 1: Preparation of Formulations According to the Invention

The following formulations have been prepared and tested for their capacity and ability to preserve myoblasts (solutions 1, 3, 4, 6, 7, 8) or mesenchymal stem cells (MSC) (A, A bis, B, C).

The myoblasts may be prepared as described in the patent application FR2810045.

The mesenchymal stem cells may be prepared as described in Sensebé L, Bourin P, Tarte K; Good manufacturing practices production of mesenchymal stem/stromal cells. Hum Gene Ther. 2011 January; 22 (1): 19-26.

Prepared Solutions (for a Final Volume of 5 mL) for the Preservation of Myoblasts

| | Reference | | 1 | | 3 | | 4 | | 5 | | 6 | | 7 | | 8 | |
|---|---|---|---|---|---|---|---|---|---|---|---|---|---|---|---|---|
| 5X Glutathione | | | | | 1000 | μL | | | | | | | | | | |
| Ion Solution * | | | | | | | | | | | | | | | | |
| 5X Ion Solution * | 1000 | μL | 1000 | μL | | | 1000 | μL | | | | | | | | |
| 5X 50% Water | | | | | | | | | 1000 | μL | | | | | | |
| Ion Solution * | | | | | | | | | | | | | | | | |
| 5X 50% Water + | | | | | | | | | | | 1000 | μL | | | 1000 | μL |
| Glutathione Ion Solution * | | | | | | | | | | | | | | | | |
| 5X Ringer Solution + | | | | | | | | | | | | | 1000 | μL | | |
| Glutathione Ion Solution * | | | | | | | | | | | | | | | | |
| Water | | | 1000 | μL | 1000 | μL | 1525 | μL | 1525 | μL | 1525 | μL | 1000 | μL | 1525 | μL |
| Plasmalyte * | 2675 | μL | 1675 | μL | 425 | μL | | | | | | | | | | |
| Ringer Solution | | | | | | | | | | | | | 425 | μL | | |
| Bicarbonate 1.4 | 1325 | μL | 1325 | μL | 1325 | μL | 1325 | μL | 1325 | μL | 1325 | μL | 1325 | μL | 1325 | μL |
| (333 mOsm/L) | | | | | | | | | | | | | | | | |
| Vitamins | | | | | 50 | μL | 50 | μL | 50 | μL | 50 | μL | 50 | μL | 50 | μL |
| (Gibco MEM Vitamin | | | | | | | | | | | | | | | | |
| Solution 100X) | | | | | | | | | | | | | | | | |
| Amino acids | | | | | 100 | μL | 100 | μL | 100 | μL | 100 | μL | 100 | μL | 100 | μL |
| (Gibco MEM | | | | | | | | | | | | | | | | |
| Amino Acid 50X) | | | | | | | | | | | | | | | | |
| Platelet Lysate *** | | | | | 1000 | μL | 1000 | μL | 1000 | μL | 1000 | μL | 1000 | μL | 1000 | μL |
| Dextran 5% | | | | | | | 250 | mg | 250 | mg | 250 | mg | | | 250 | mg |
| Glutamax ** | | | | | 1000 | μL | | | | | 100 | μL | 100 | μL | 100 | μL |
| (100X) Liquid | | | | | | | | | | | | | | | | |
| Total | 5000 | μL | 5000 | μL | 5000 | μL | 5000 | μL | 5000 | μL | 5000 | μL | 5000 | μL | 5000 | μL |

| | | | | | | | | |
|---|---|---|---|---|---|---|---|---|
| pH at 1 h, (+3 ± 2° C.) | 7.67 | 7.49 | 7.33 | 7.41 | 7.52 | 7.38 | 7.9 | 7.81 |

Prepared Solutions (Final Volume of 10 ml) for
Preservation of Mesenchymal Stem Cells (MSC)

| | sol A | | sol A bis | | sol B | | sol C | |
|---|---|---|---|---|---|---|---|---|
| 5X Ringer Solution + | 2000 | μL | 2000 | μL | 2000 | μL | 2000 | μL |
| Glutathione Ion Solution * | | | | | | | | |
| Water | 2000 | μL | 2000 | μL | 3000 | μL | 2600 | μL |
| Ringer Solution * | 850 | μL | 850 | μL | 1850 | μL | 1450 | μL |
| Liquid Bicarbonate | 2650 | μL | 2650 | μL | 2650 | μL | 2650 | μL |
| 1.4% (333 mOsm/L) | | | | | | | | |
| Vitamins | 100 | μL | 100 | μL | 100 | μL | 100 | μL |
| (Gibco MEM Vitamin | | | | | | | | |
| Solution 100X) | | | | | | | | |
| Amino acids | 200 | μL | 200 | μL | 200 | μL | 200 | μL |
| (Gibco MEM | | | | | | | | |
| Amino Acid 50X) | | | | | | | | |
| Platelet Lysate *** | 2000 | μL | | | | | 800 | μL |
| Irradiated Platelet | | | 2000 | μL | | | | |
| Lysate *** | | | | | | | | |
| Glutamax | 200 | μL | 200 | μL | 200 | μL | 200 | μL |
| (100X) Liquid** | | | | | | | | |
| Total | 10000 | μL | 10000 | μL | 10000 | μL | 10000 | μL |

| | | | | |
|---|---|---|---|---|
| pH at 1 h, | 8.07 | 7.93 | 7.73 | 7.78 |
| (+3 ± 2° C.) | | | | |

* See below
**200 mM L-alanyl-L-glutamine mixture
*** Platelet Lysate Composition

The ingredient “5× Ion Solution” corresponds to:

| | 5X Ion Solution | |
|---|---|---|
| | (for 20 mL of Solution) | |
| Plasmalyte (see below) | 20 | mL |
| Magnesium | 32.5 | mg |
| Glucose | 270.3 | mg |
| Sodium Acetate | 172.3 | mg |
| Trisodium Citrate | 276.5 | mg |
| Citric Acid | 92.2 | mg |

The ingredient “5× Glutathione Ion Solution” corresponds to:

| | 5X Glutathione Ion Solution | |
|---|---|---|
| | (for 20 mL of Solution) | |
| Plasmalyte (see below) | 20 | mL |
| Magnesium | 32.5 | mg |
| Glucose | 270.3 | mg |
| Sodium Acetate | 172.3 | mg |
| Trisodium Citrate | 276.5 | mg |
| Citric Acid | 92.2 | mg |
| Glutathione Reduced | 33.8 | mg |

The ingredient “5×50% Water Ion Solution” corresponds to:

| | 5X 50% Water Ion Solution | |
|---|---|---|
| | (for 20 mL of Solution) | |
| Water for injectable preparation | 10 | mL |
| (water for injection WFI) | | |
| Plasmalyte (see below) | 10 | mL |
| Magnesium | 32.5 | mg |
| Glucose | 270.3 | mg |
| Sodium Acetate | 172.3 | mg |
| Trisodium Citrate | 276.5 | mg |
| Citric Acid | 92.2 | mg |

The ingredient “5×50% Water+Glutathione Ion Solution” corresponds to:

| | 5X 50% Water + | |
|---|---|---|
| | Glutathione Ion Solution | |
| | (for 20 mL of Solution) | |
| Water for injectable preparation | 10 | mL |
| (water for injection WFI) | | |
| Plasmalyte (see below) | 10 | mL |
| Magnesium | 32.5 | mg |
| Glucose | 270.3 | mg |
| Sodium Acetate | 172.3 | mg |
| Trisodium Citrate | 276.5 | mg |
| Citric Acid | 92.2 | mg |
| Glutathione Reduced | 33.8 | mg |

The ingredient “5× Ringer Solution+Glutathione Ion Solution” corresponds to:

| | 5X Ringer Solution + | |
|---|---|---|
| | Glutathione Ion Solution | |
| | (for 20 mL of Solution) | |
| Ringer Solution (see below) | 20 | mL |
| Magnesium | 32.5 | mg |
| Glucose | 270.3 | mg |
| Sodium Acetate | 172.3 | mg |
| Trisodium Citrate | 276.5 | mg |
| Citric Acid | 92.2 | mg |
| Glutathione Reduced | 33.8 | mg |

The ingredient “Plasmalyte” corresponds to:

| | Plasmalyte |
|---|---|
| | (for 1000 mL of Solution) |
| NaCl | 5.26 g |
| KCl | 0.37 g |
| MgCl2 | 0.30 g |
| Sodium Acetate Trihydrate | 3.68 g |
| (C2H3NaO2) | |
| Sodium Gluconate | 5.02 g |
| Water for injectable preparation | Qsp (qty sufficient for) 1000 mL |
| (water for injection WFI) | |

The ingredient “Ringer Solution” corresponds to:

| | Ringer Solution |
|---|---|
| | (for 1000 mL of Solution) |
| NaCl | 8.60 g |
| KCl | 0.30 g |
| Calcium Chloride Dihydrate | 0.33 g |
| Water for injectable preparation | Qsp (qty sufficient for) 1000 mL |
| (water for injection WFI) | |

*** Composition of platelet lysate growth factors:

| | Concentration | Relative Standard |
|---|---|---|
| Growth Factor | in pg/mL | Deviation (%) |
| PDGF-AB | 60000 | 8.39 |
| IGF-1 | 49000 | 3.88 |
| EGF | 3500 | 4.79 |
| VEGF | 910 | 4.22 |
| bFGF | 150 | 6.33 |

### Example 2: Preservation of Myoblasts in the Formulations of the Invention

Experimental Protocol:

The viability of the myoblasts was measured according to the protocol below:

The viability measurement by flow cytometry is carried out after labelling of the cells with propidium Iodide (PI) which is a marker of cell viability. It provides the ability to determine the viability of the cells.

The measurement of the percentage of myoblasts is carried out by flow cytometry. It corresponds to the percentage of live cells PI−, CD56+, CD15− after labelling of the cells by using specific antibodies CD56, CD15 and PI. In fact, the product tested also contains impurities which are CD56− and CD15 positive or negative cells and which can prevail over the myoblasts because they are less demanding in terms of culturing. The goal is to maintain the percentage of myoblasts in the product after freezing and therefore to get closer to the value before freezing.

The cell viability and the measurement of the percentage of myoblasts were analysed directly prior to formulation (T0h) then 24, 48, 72 or 144 hours after formulation and incubation at +3±2° C.

Results:

1^{st} Series of Tests

| | | Solution | Solution | Solution | Solution | Solution |
|---|---|---|---|---|---|---|
| | Reference | 1 | 3 | 4 | 5 | 6 |
| Viability | 99% | | | | | |
| T0 h | | | | | | |
| Viability | 77.9% | 84.1% | 97.1% | 92.5% | 92.5% | 92.6% |
| T24 h | | | | | | |
| Viability | 48.2% | 55.0% | 92.6% | 64.5% | 63.8% | 65.2% |
| T48 h | | | | | | |
| Viability | 9.7% | 19.0% | 84.6% | 25.4% | 26.9% | 29.0% |
| T72 h | | | | | | |

2^{nd} Series of Tests

| | | Solution 3 | | |
|---|---|---|---|---|
| | Solution | (prepared in | Solution | Solution |
| | 3 | duplicate) | 7 | 8 |
| Viability T0 h | 99.4 | | | |
| Viability T24 h | 94.2% | 92.9% | 93.9% | 91.3% |
| Viability T48 h | 82.4% | 82.9% | 82.8% | 65.7% |
| Viability T72 h | 73.0% | 73.8% | 74.8% | 31.6% |
| % Myoblasts T72 h | 46.1% | 45.4% | 44.7% | |
| Viability T144 h | 64.0% | 64.2% | 68.1% | |
| % Myoblasts T144 h | 52.6% | 55.6% | 53.3% | |

3^{rd} Series of Tests

| | Solution 3 | Solution 7 |
|---|---|---|
| Viability T24 h | 87.4% | 88.8% |
| Viability T48 h | 83.5% | 87.6% |
| Viability T72 h | 70.6% | 81.4% |

It has evidently emerged that:
the formulations prepared have a physiological pH of between 7 and 8.5;
the formulations have an osmolarity of between 300 and 400 mOsm/L, approaching physiological osmolarity;
the formulations 3 and 7 used for test series no 3, prepared 6 days in advance and conserved at +3±2° C., are stable and indeed preserve the cells;
the addition of 5% dextran causes some stress for the cells and appears to be involved in inducing a decrease in cell viability thereof (formulations 4 to 6);
the formulations containing glutathione better preserve the cells at +3±2° C.

The viability values for the cells obtained after conservation thereof for 72 hours in the formulations comprising glutathione are greater than 70% (see formulations 3 and 7);
the use of Plasmalyte or Ringer solution does not alter the solution (see formulations 3 and 7);
the most efficacious formulations seem to be formulation 3 or 7 (Plasmalyte or Ringer solution formulation, without dextran and with glutathione). They serve to enable the preservation of: more than 85% of the cells after conservation thereof for 24 hours at +3±2° C.; more than 80% of the cells after conservation thereof for 48 hours at +3±2° C.; and more than 70% of the cells after conservation thereof for 72 hours at +3±2° C.

### Example 3: Preservation of Mesenchymal Stem Cells in the Formulations of the Invention

a) Cell Viability Test

Experimental Protocol:

To determine the viability of the MSCs in the solutions tested, the cells were first concentrated to 2000 cells/μL and then diluted 1:2 with trypan blue (marker of cell mortality). A volume of 1 mm³ of cells diluted in the trypan blue solution is deposited on a Malassez chamber and the cells are counted under the microscope (objective lens 40×). The dead cells are marked with trypan blue while the live cells are able to release it and are therefore not stained.

The computation of cell concentration and cell viability is done according to the following calculation:

$$Concentration = \frac{No of cells counted \times 100 \times 1000 \times dilution factor}{No of squares counted} ( cells / mL )$$

$$\% viability = \frac{Live cells}{live + dead cells}$$

When the cells are formulated in the solutions A, A bis, B and C, they are conserved for 72 hours at +3±2° C., and thereafter the cell viability is measured.

The “DP AH4%” corresponds to the reference solution in which the cells are formulated only in 4% human albumin and conserved for 24 hours at +3±2° C.

The Drug Substance “DS” corresponds to the cells obtained from the harvest at the end of the process prior to formulation.

Results:

| | | Time | % Viability with |
|---|---|---|---|
| MSC Batch | Formulation | Period | Trypan Blue |
| MSC 1 | DS | T0 h | 94 |
| | DP AH4% | T24 h | 60.6 |
| | Sol A | T72 h | 83.8 |
| | Sol B | T72 h | 86.5 |
| MSC 2 | DS | T0 h | 93.5 |
| | DP AH4% | T24 h | 71.7 |
| | Sol A | T72 h | 84.9 |
| | Sol B | T72 h | 77.9 |
| | Sol C | T72 h | 71.8 |
| MSC 3 | DS | T0 h | 100 |
| | DP AH4% | T24 h | 65.2 |
| | Sol A | T72 h | 75.8 |
| | Sol A bis | T72 h | 79.5 |
| | Sol B | T72 h | 78.1 |

It has evidently emerged that the solutions A, A bis, B and C serve to enable good preservation of the cells formulated in these solutions after conservation thereof for 72 hours at +3±2° C. (more than 70% cell viability for each of these solutions).

b) Phenotype

Experimental Protocol:

An analysis of the phenotype of the cells is carried out in order to determine the stability of the MSCs in formulation in the solutions A, A bis, B and C. The phenotype is analysed by means of flow cytometry and corresponds to the percentage of CD90+/CD73+/CD45−/CD34− cells, by using specific antibodies CD90, CD73, CD45 and CD34 labelled with Phycoerythrin (PE).

When the cells are formulated in the solutions A, A bis, B and C, they are conserved for 72 hours at +3±2° C. At 72 hours, observation of the phenotype of the cells is performed.

The Drug Substance “DS” corresponds to the cells obtained from the harvest at the end of the process prior to formulation.

Results:

| | | | Marker | | | |
|---|---|---|---|---|---|---|
| | | | CD90 | CD73 | CD45 | CD34 |
| MSC | | Time | Spec ≥ | Spec ≥ | Spec ≤ | Spec ≤ |
| Batch | Formulation | Period | 95% | 95% | 5% | 2% |
| MSC 1 | DS | T0 h | 99.8 | 99.7 | 0.1 | 0.1 |
| | Sol A | T72 h | 99.8 | 99.2 | 0.4 | 0.2 |
| | Sol B | T72 h | 99.6 | 99.4 | 0.5 | 0.3 |
| MSC 2 | DS | T0 h | 99 | 99.5 | 0.5 | 0.3 |
| | Sol A | T72 h | 95.9 | 97.7 | 0.1 | 0 |
| | Sol B | T72 h | 99.2 | 92.6 | 0.2 | 0 |
| | Sol C | T72 h | 97.7 | 97.4 | 0.2 | 0 |
| MSC 3 | DS | T0 h | 99.3 | 99.1 | 0.3 | 0.3 |
| | Sol A | T72 h | 99.9 | 99.7 | 0.4 | 0.2 |
| | Sol A bis | T72 h | 99.8 | 97.2 | 0.2 | 0.1 |
| | Sol B | T72 h | 99.8 | 96.1 | 0.8 | 0.8 |

The phenotype of the MSCs formulated in the solutions A, A bis, B and C is indeed well preserved after 72 hours of being conserved at +3±2° C. (more than 95% of the markers CD90 and CD73, less than 2% of the markers CD45 and CD34).

c) Apoptosis Test

Experimental Protocol:

The apoptosis test is used to determine premature cell death by apoptosis.

The principle of the apoptosis test is based on SYTOX green double labelling (membrane integrity marker—dead cell marker), Annexin V (early apoptosis marker) analysed by flow cytometry. This labelling makes it possible to distinguish the cells in early apoptosis (SYTOX−/AnnexinV+), from the dead cells (SYTOX+/AnnexinV+) and live cells (SYTOX−/AnnexinV−).

When the cells are formulated in the solutions A, A bis, B and C, they are conserved for 72 hours at +3±2° C. At 72 hours, the apoptosis test is subsequently carried out.

The “DP AH4%” corresponds to the reference solution in which the cells are formulated only in 4% human albumin and conserved for 24 hours at +3±2° C.

The Drug Substance “DS” corresponds to the cells obtained from the harvest at the end of the process prior to formulation.

Results

| | | | % of Non- | |
|---|---|---|---|---|
| | | Time | Apoptotic | % Deviation from |
| Batch Number | Formulation | Period | Cells | DP AH 4% |
| MSC 1 | DS | T0 | 95.5 | |
| | DP AH4% | T24 h | 0.2 | |
| | Sol A | T72 h | 85.8 | +428% |
| | Sol B | T72 h | 69.2 | +345% |
| MSC2 | DS | T0 | 85.9 | |
| | DP AH4% | T24 h | 51.9 | |
| | Sol A | T72 h | 84.9 | +12.7% |
| | Sol B | T72 h | 77.9 | +17.7% |
| | Sol C | T72 h | 71.8 | +3.5% |
| MSC 3 | DS | T0 | 89.1 | |
| | DP AH4% | T24 h | 19.8 | |
| | Sol A | T72 h | 57.6 | +191% |
| | Sol A bis | T72 h | 58.9 | +197% |
| | Sol B | T72 h | 39.3 | +98% |

The percentage of non-apoptotic cells after conservation of cells for 72 hours at +3±2° C. for the formulations prepared with the solutions A, A bis, B and C is greater than the percentage of non-apoptotic cells for the cells formulated in the reference solution (DP AH4%) after 24 hours at +3±2° C.

It has evidently emerged from these analyses that:
the prepared formulations A, A bis, B and C are stable and indeed preserve well the phenotype of the MSCs for 72 hours at +3±2° C.;
the prepared formulations A, A bis, B and C serve to enable good preservation of the cells (more than 70% of viable cells after conservation thereof for 72 hours at +3±2° C.).

1. A composition comprising, in a physiologically acceptable medium:
a) at least one saccharide;
b) at least one vitamin;
c) at least one amino acid;
d) at least one antioxidant, and
e) cells for therapeutic use;
said composition having a pH of between 7.0 and 8.5.

2. The composition according to claim 1, wherein it has a pH between 7.0 and 8.3.

3. The composition according to claim 1, wherein it comprises a platelet lysate.

4. The composition according to claim 1, wherein the saccharide is selected from among monosaccharides, disaccharides and trisaccharides.

5. The composition according to claim 1, wherein the vitamin is selected from among the vitamins B1, B2, B4, B5, B6, B7, B9, PP and the mixtures thereof.

6. The composition according to claim 1, wherein the amino acid is selected from among glutamine, alanyl-glutamine, tryptophan, lysine, methionine, phenylalanine, threonine, valine, leucine and isoleucine, arginine, histidine, tyrosine, cysteine and the mixtures thereof.

7. The composition according to claim 1, wherein the amino acid is cysteine.

8. The composition according to claim 1, wherein the antioxidant is selected from among glutathione, vitamin C, vitamin E, vitamin A, L-cysteine, or the coenzyme Q10.

9. The composition according to claim 1, wherein the antioxidant is glutathione.

10. The composition according to claim 1, wherein it comprises at least one bicarbonate salt.

11. The composition according to claim 1, wherein the cells for therapeutic use are selected from among: natural killer (NK) cells; monocytes; B lymphocytes; T lymphocytes, whether natural or genetically modified; myoblasts; hematopoietic stem cells; mesenchymal stem cells; cardiac cells; and fibroblasts.

12. The composition according to claim 1, wherein the physiologically acceptable medium is an aqueous medium containing electrolytes, and wherein the composition comprises:
a) glucose;
b) a mixture of vitamins B1, B2, B4, B5, B6, B7, B9 and PP;
c) a mixture of glutamine, alanyl-glutamine, tryptophan, lysine, methionine, phenylalanine, threonine, valine, leucine, isoleucine, arginine, histidine, tyrosine and cysteine;
d) glutathione; and
e) cells for therapeutic use.

13. A cell preservation method for preserving a sample of cells for therapeutic use, comprising at least one mixing step of mixing the sample of cells for therapeutic use with:
a) at least one saccharide;
b) at least one vitamin;
c) at least one amino acid;
d) at least one antioxidant; and
a physiologically acceptable medium;
the composition thus obtained having a pH between 7.0 and 8.5.

14. The cell preservation method according to claim 13, wherein the pH is between 7.0 and 8.3.

15. A method for preserving at least one sample of cells for therapeutic use, comprising mixing said sample of cells with a composition comprising, in a physiologically acceptable medium:
a) at least one saccharide;
b) at least one vitamin;
c) at least one amino acid;
d) glutathione;
and having a pH of between 7.0 and 8.5.

16. The method according to claim 15, said composition having a pH of between 7.0 and 8.3.

17. The composition according to claim 2, wherein it comprises a platelet lysate.

18. The composition according to claim 2, wherein the saccharide is selected from among monosaccharides, disaccharides and trisaccharides.

19. The composition according to claim 3, wherein the saccharide is selected from among monosaccharides, disaccharides and trisaccharides.

20. The composition according to claim 2, wherein the vitamin is selected from among the vitamins B1, B2, B4, B5, B6, B7, B9, PP and the mixtures thereof.
